(19) United States
(12) Patent Application Publication
(10) Pub. No.: US 2024/0252042 A1
(43) Pub. Date: Aug. 1, 2024

(54) TISSUE EVALUATION FOR APNEA THERAPY

(71) Applicant: MAGNAP, INC., San Francisco, CA (US)

(72) Inventors: Joseph LAMBERTI, Castro Valley, CA (US); Michael HARRISON, San Francisco, CA (US); Michael DANTY, Santa Rose, CA (US)

(73) Assignee: MAGNAP, INC., San Francisco, CA (US)

(21) Appl. No.: 18/422,362

(22) Filed: Jan. 25, 2024

Related U.S. Application Data
(60) Provisional application No. 63/481,898, filed on Jan. 27, 2023.

(51) Int. Cl. A61B 5/00; A61B 5/11

(52) U.S. Cl. CPC A61B 5/0057; A61B 5/11; A61B 2562/0247

(57) ABSTRACT

Tissue force displacement data can be used to evaluate the suitability of a subject for an apnea therapy, such as therapy with an implanted magnet. A method of evaluating a suitability of a subject for an apnea therapy comprises placing an engagement structure on a tissue of the subject to engage the tissue of the subject. The engagement structure is moved toward the tissue to a location to move the tissue. One or more of the location or a force to the engagement structure at the location is measured. The suitability of the of the subject for the apnea therapy is determined in response to the force at the location.

### RELATED APPLICATIONS

This application claims the benefit under 35 U.S.C. § 119(e) of U.S. Provisional Patent Application No. 63/481,898, filed Jan. 27, 2023, which is incorporated, in its entirety, by this reference.

### BACKGROUND

Obstructive sleep apnea (OSA) is a condition in which a subject's airway collapses during sleep. This condition can make it difficult for people to sleep, and people with OSA can be somewhat sleep deprived in at least some instances.

The prior approaches to treating OSA can be less than ideal in at least some respects. Several therapies have been proposed for OSA, such as continuous positive airway pressure (CPAP), mandibular advancement devices (MAD), and implantable magnets. Although somewhat effective, CPAP typically relies on a face mask and positive pressure and some subjects can become intolerant of wearing a mask on their face at night and may feel somewhat claustrophobic in at least some instances. Also, CPAP systems can be somewhat larger than would be ideal. Although MAD devices have been proposed, advancing the mandible can be a somewhat indirect approach to treating airway obstruction and can be somewhat uncomfortable for at least some subjects. A magnet implanted on the hyoid bone has been proposed to prevent airway collapse, in which the implanted magnet couples to an external magnet to urge the hyoid bone and associated tissue anteriorly to prevent obstruction of the airway. Although initial clinical studies with this approach are encouraging, work in relation to the present disclosure suggests that the efficacy of this approach may not less than ideally suited for some apnea patients.

Clinical studies have been performed to determine which therapies might be suitable for some subjects. For example, cephalometric analysis of radiographic images has been used to determine the relative position of the hyoid bone, the mandibular plane and other anatomical structures, and this data has been used to evaluate mandibular advancement devices. However, this approach is somewhat limited because the elasticity and associated stiffness of tissue is not measured with this approach. Work in relation to the present disclosure suggests that the efficacy of the above approaches may be related to tissue elasticity and stiffness.

In light of the above, it would be helpful to incorporate data related to tissue stiffness and elasticity with other data such as polysomnography data to determine the suitability of a subject for type of OSA therapy.

### SUMMARY

In some embodiments, tissue force displacement data is used to evaluate the suitability of a subject for an apnea therapy, such as therapy with an implanted magnet.

In a first aspect, method of evaluating a suitability of a subject for an apnea therapy comprises placing an engagement structure on a tissue of the subject to engage the tissue of the subject. The engagement structure is moved toward the tissue to a location to move the tissue. One or more of the location or a force to the engagement structure at the location is measured. The suitability of the of the subject for the apnea therapy is determined in response to the force at the location.

In a second aspect, a system for evaluating a suitability of a subject for an apnea therapy comprises an engagement structure sized and shaped to engage a tissue of the subject, an extension coupled to the engagement structure, and a force sensor coupled to the extension and the engagement structure to measure a force to the tissue of the subject. A movement sensor is coupled to the extension and the engagement structure to measure an amount of displacement of the engagement structure. A processor is coupled to the force sensor and the movement sensor to measure the force to the tissue and an amount of displacement.

In another aspect, a method of evaluating suitability of a subject for an apnea therapy comprises receiving force displacement data from tissue of the subject and determining the suitability of the subject in response to the force displacement data.

### INCORPORATION BY REFERENCE

All patents, applications, and publications referred to and identified herein are hereby incorporated by reference in their entirety and shall be considered fully incorporated by reference even though referred to elsewhere in the application.

### BRIEF DESCRIPTION OF THE DRAWINGS

A better understanding of the features, advantages and principles of the present disclosure will be obtained by reference to the following detailed description that sets forth illustrative embodiments, and the accompanying drawings of which:

FIGS. 1 and 2 show a tissue model for apnea treatment with a magnetic device configured to move the hyoid bone anteriorly, in accordance with some embodiments;

FIG. 3 shows a tissue model with a force to the neck directed posteriorly, in accordance with some embodiments;

FIG. 4 shows a system to measure force displacement data of the neck, in accordance with some embodiments;

FIG. 5 shows a tissue engagement structure, in accordance with some embodiments;

FIG. 6 shows a system to measure force displacement data of tissue such as neck tissue, in accordance with some embodiments;

FIG. 7 shows a method of evaluating suitability of a subject for an apnea therapy, in accordance with some embodiments; and

FIG. 8 shows force and displacement data for a subject, in accordance with some embodiments.

### DETAILED DESCRIPTION

The following detailed description provides a better understanding of the features and advantages of the inventions described in the present disclosure in accordance with the embodiments disclosed herein. Although the detailed description includes many specific embodiments, these are provided by way of example only and should not be construed as limiting the scope of the inventions disclosed herein.

The presently disclosed systems, methods and apparatus are well suited for use with many types of prior sleep apnea therapies such as CPAP, MAP and the Magnap device.

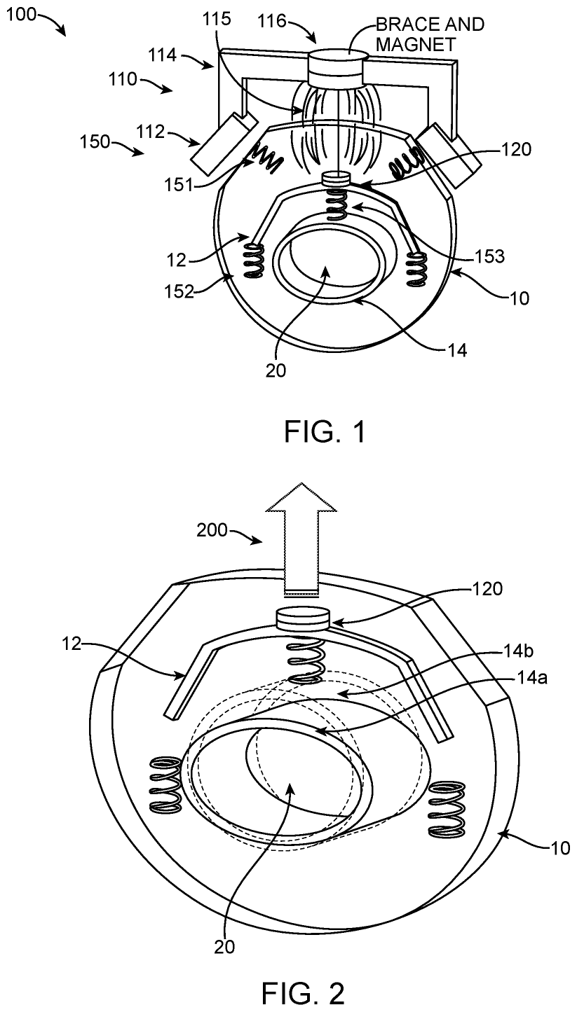

FIGS. 1 and 2 show a tissue model for apnea treatment with a magnetic device 100 configured to move the hyoid bone 12 anteriorly with a magnetic implant 120. The hyoid bone 12 is completely suspended in the neck 10 by muscles and ligaments. The hyoid bone also has connections to the back of the tongue and airway. By applying anteriorly directed forces to the hyoid bone, sleep apnea can be decreased in at least some subjects.

The magnetic implant 120 is configured to couple to an external magnet 114 to urge the hyoid bone anteriorly with magnetic field 115. The magnetic implant 120 can be coupled to the bone in any suitable way, for example with a clip. The magnetic implant 120 may comprise any suitable magnetic material, such as a magnet or ferromagnetic material, for example. The system 100 comprises an external brace 110 to support external magnet 116. The external brace 110 may comprise a neck engaging portion 112 configured to engage the neck from each lateral side of the neck. The external brace 110 may comprise one or more arms to 114 extending between the neck engaging portion 112 and external magnet 116, in order to suspend external magnet 116 above the neck.

In some embodiments, efficacy can be related to the elasticity of tissues. In some embodiments, the stiffness and elasticity of the neck tissues correspond to elements of a model 150. The model 150 may comprise corresponding stiffnesses and elasticities to model movement of the hyoid bone and related tissue structures. In some embodiments, the model 150 comprises the stiffness and elasticity 151 of the tissue that supports the brace, the stiffness and elasticity 152 of the tissue that supports the hyoid bone, and the stiffness and elasticity 153 of the connection between the hyoid bond and the airway 20 through the throat tissue, such as one or more of the trachea or the larynx 14.

In some embodiments, the magnetic device 100 comprises one or more components of the Magnap device undergoing clinical trials. In some embodiments, the magnetic implant 120 comprises a neodymium-iron-boron rare earth magnet with a ferromagnetic directional back-plate encased in titanium. In some embodiments, the subject is fitted with a custom, removable external brace 110 comprising the second magnet 116, which is worn during sleep and configured to prevent airway collapse by attracting the internal hyoid magnet with sufficient force to keep the airway open.

As shown in FIG. 2 the interaction between the external magnet and implanted magnet 120 provides an anteriorly directed force 200 so as to displace the hyoid bone 12 anteriorly. In some embodiments the throat tissue, such as one or more of the trachea or the larynx 14, comprises a first configuration 14a without force 200, and a second configuration 14b in which the airway 20 is enlarged.

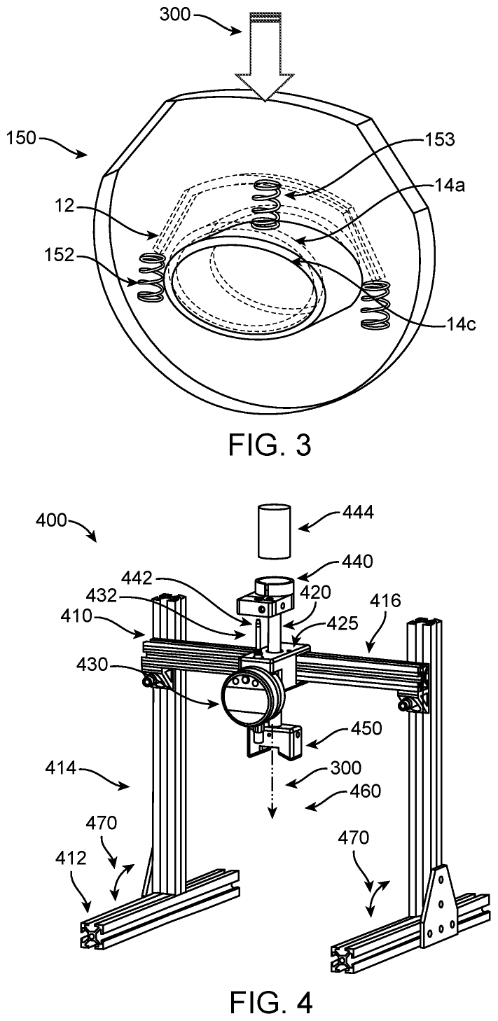

FIG. 3 shows the tissue model 150 with a force 300 to the neck directed posteriorly. In some embodiments, the force displacement data of force 300 directed posteriorly is related to the force displacement data of force 200 of the hyoid bone directed anteriorly. In some embodiments, this relationship allows the force displacement data for the force 300 directed posteriorly to be used to predict the anterior movement of the hyoid bone anteriorly and associated efficacy in treating sleep apnea. In some embodiments, movement of the hyoid bone 12 posteriorly results in a change to tissues associated with model 150, such as from the first configuration 14a to second configuration 14c of the throat tissue such as one or more of the trachea or the larynx. In some embodiments, the stiffness and elasticity 152 of the tissues supporting the hyoid bone are related to movement of the hyoid bone, and the stiffness and elasticity 153 of the connection between the hyoid and the airway.

FIG. 4 shows a system 400 to measure force displacement data of the neck. The system 400 comprises a support 410 coupled to a tissue engagement structure 450 and force displacement sensor 430. The force displacement sensor 430 is configured to measure one or more of force data or displacement data while the engagement structure 450 contacts tissue, such as skin tissue on a neck of a subject.

The support 410 comprises a base 412, an upwardly extending arm 414 and a laterally extending arm 416. The base 412 is coupled to the upwardly extending arm 414 and the laterally extending arm 416 to support the force displacement sensor 430. The laterally extending arm 416 is coupled to a coupling 425. In some embodiments, the coupling 425 comprises a guide, such as a channel, which allows the extension 420 to slide there through. In some embodiments, the coupling 425 is coupled to the force displacement sensor 430 with a fixed relationship, while a portion of the force displacement sensor moves in response to movement of the extension 420 and engagement structure 450.

An extension 420 is coupled to the engagement structure 450 and the force displacement sensor 430. The extension 420 is sized to allow translation displacement of the neck engagement structure 450 along a movement axis 460, such as posterior translational movement. In some embodiments, movement axis 460 is aligned with an elongate axis of extension 420. The extension 420 is coupled to one or more structures 440 configured to engage force displacement sensor 430 to acquire force displacement data. In some embodiments, the one or more structures 430 comprises a surface 430 to engage a portion 432 of sensor 430 to provide displacement data.

While the system 400 can be configured in many ways, in some embodiments system 400 is configured to measure displacement along axis 460 in response to force applied to the neck. In some embodiments, a weight 444 is loaded onto the one or more structures 440, such as a coupling sized to receive the weight. Weights of increasing or decreasing weight can be added, and the position of the engagement structure and corresponding movement of the tissue such as neck tissue determined. In some embodiments, the force displacement sensor comprises a micrometer configured to measure a position of surface 442 engaging the portion 432, which may comprise an extension of a digital micrometer. The amount of displacement along axis 460 for a provided amount of weight 444 can be used to determine the elasticity of neck tissue.

In some embodiments, the system 400 is configured to adjust the measurement direction in an inferior superior direction, e.g. a head to toe direction. While this can be accomplished in many ways, in some embodiments one or more components of the support 410 is configured to pivot relative to the base 412 as shown with arrows 470. In some embodiments a lockable hinge or other pivoting structure couples base 412 to support 410 so as to allow the measurement direction to be adjusted with rotation of the force displacement sensor 430 and the engagement structure 450 relative to base 412. In some embodiments, the measurement direction is adjusted by rotating the force displacement sensor 430 relative to the support 410, for example.

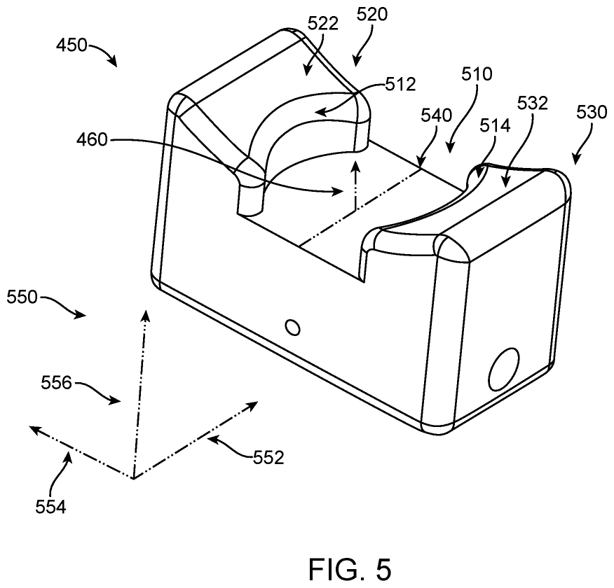

FIG. 5 shows a tissue engagement structure 450. In some embodiments, engagement structure 450 is configured to engage the skin of a neck of a subject with advancement in an anterior to posterior direction along axis 460. The tissue engagement structure comprises a recess 510. In some embodiments, the recess 510 is sized to receive at least a portion of a laryngeal prominence of a subject. In some embodiments, the engagement structure 450 comprises a midline 540 configured to be aligned with a midline of the subject extending in an inferior superior direction, for example aligned with a sagittal plane of the subject. The engagement structure 450 comprises a first protrusion 520 on a first side and a second protrusion 530 on a second side of the engagement structure 450. In some embodiments, the first protrusion 520 comprises a first surface 522 to engage the skin of the subject on a first side of the neck, and the second protrusion 520 comprises a second surface 532 to engage the skin of the neck of the subject on a second side of the neck.

While the recess 510 can be configured in many ways, in some embodiments, the recess 510 comprises a first concave portion 512 on a first side and a second concave portion 514 on a second side. In some embodiments, the first concave portion 512 is defined with a portion of protrusion 520 and the second concave portion 514 is defined with a portion of protrusion 530.

In some embodiments, the engagement structure 450 corresponds to 3 reference axes 550 of the subject. In some embodiments, a first axis 552 corresponds to an inferior superior direction of the subject, second axis 554 corresponds to lateral directions on the subject, and a third axis 556 corresponds to an inferior superior direction of the subject.

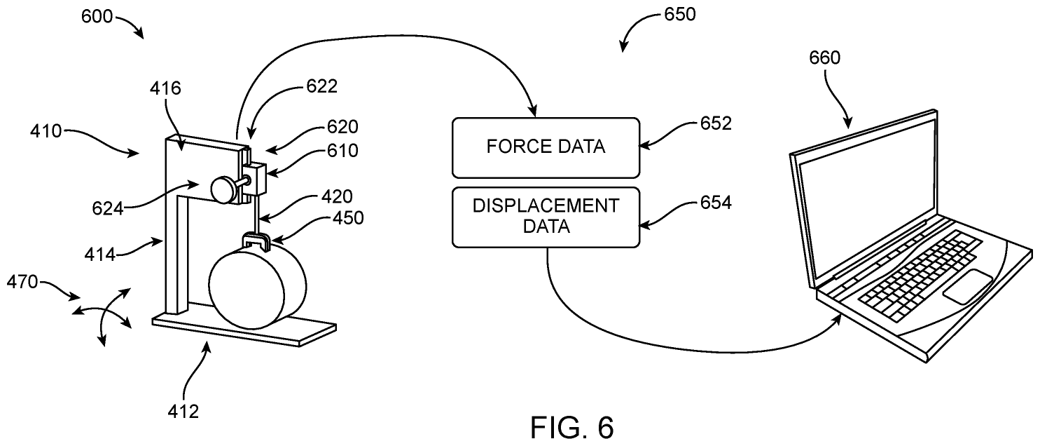

FIG. 6 shows a system 600 to measure force displacement data of tissue. The system 600 may comprises one or more components of system 400 described herein with reference to FIG. 4, such as base 412, upwardly extending arm 414, laterally extending arm 416, extension 420 and tissue engagement structure 450. In some embodiments, a force displacement sensor 610 comprises a displacement sensor to measure translation of extension 420 and a force sensor to measure force from the extension 420 coupled to the engagement structure 450 in contact with the tissue. In some embodiments, the force displacement sensor comprises a linkage 620, such as a rack 622 and pinion that is configured to translate the extension 420 and engagement structure 450 with rotation of a knob 624. A processor 660 is configured to receive data 650, such as force data 652 and displacement data 654 for a plurality of displacement locations. The force displacement sensors may comprise one or more components of a commercially available force displacement sensor, such as one or more components of as the FSA-MSL Portable Force/Displacement Tester, commercially available from Imada, Inc. (imada.com), for example.

In some embodiments, the system 400 is configured to adjust the measurement direction in an inferior superior direction, e.g. a head to toe direction. While this can be accomplished in many ways, in some embodiments one or more components of the support 410 is configured to pivot relative to the base 412 as shown with arrows 470. In some embodiments a lockable hinge or other pivoting structure couples base 412 to support 410 so as to allow the measurement direction to be adjusted with rotation of the force displacement sensor 430 and the engagement structure 450 relative to base 412. Alternatively or in combination, the measurement system 400 can be configured to adjust the measurement direction laterally, e.g. side to side relative to the patient. In some embodiments, the measurement system 400 is configured to adjust the measurement system 400 in an inferior superior direction and laterally, for example with a movable mount such as a gimbal. In some embodiments, the measurement direction is adjusted by rotating the force displacement sensor 430 relative to the support 410, for example.

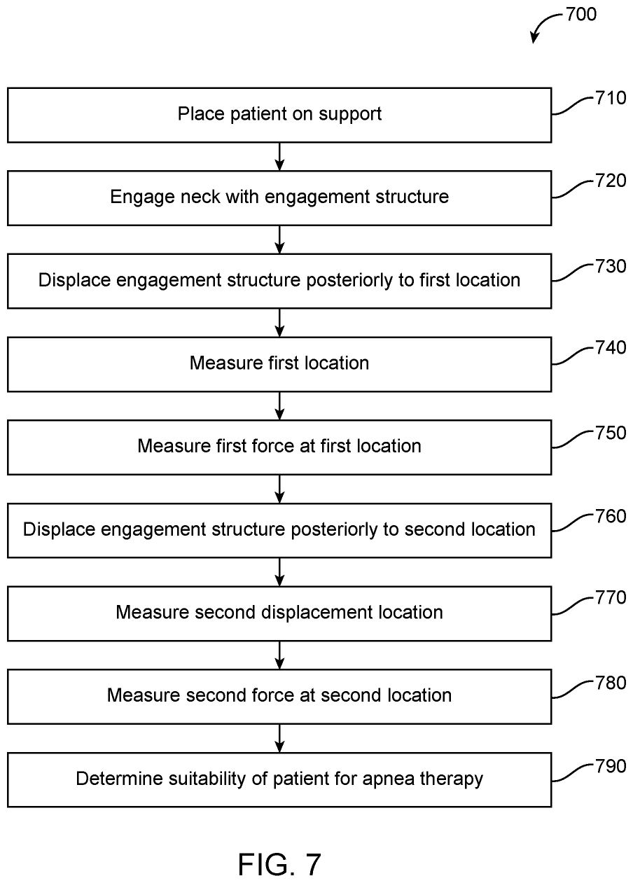

FIG. 7 shows a method 700 of evaluating suitability of a subject for an apnea therapy.

At a step 710, the patent is placed on a support.

At a step 720, the neck is engaged with an engagement structure.

At a step 730, the engagement structure is displaced posteriorly to first location

At a step 740, the first displacement location is measured.

At a step 750, a first force is measured at the first displacement location.

At a step 760, the engagement structure is displaced posteriorly to a second location.

At a step 770, the second displacement location is measured

At a step 780, a second force is measured at the second displacement location

At a step 790, the suitability of the subject for apnea therapy is determined.

Although method 700 describes a method of evaluating suitability of a subject for an apnea therapy in accordance with some embodiments, one of ordinary skill in the art will recognize many adaptations and variations. One or more of the steps can be removed, some of the steps repeated, and the steps can be performed in any order. Some of the steps may comprise sub steps of other steps.

### Experimental

The present inventor conducted experiments to measure force displacement data with an engagement structure and force displacement sensor in accordance with the embodiments disclosed herein. The amount of force varied from 0 Newtons (N) to 15 N. The movement of the extension and engagement structure were measured when the force was applied. While the data can be analyzed in many ways, Table 1 shows N per mm data for the subjects for slope data from 2 N to 3 N, and for 9 N to 10 N. For the 2 N to 3 N segment, the values ranged from 0.192 N/mm to 0.741 N/mm. For the 9 N to 10 N segment, the values ranged from 1.590 N/mm to 2.277 N/mm.

TABLE 1
Force displacement data measured from 7 subjects

| | Slope (Segment 2 N-3 N) | Slope (Segment 9 N-10 N) |
|---|---|---|
| Subject | (N/mm) | (N/mm) |
| 1 | 0.517 | — |
| 2 | 0.192 | — |
| 3 | 0.391 | 1.590 |
| 4 | 0.741 | 1.767 |
| 5 | 0.518 | 1.895 |
| 6 | 0.401 | 2.277 |
| 7 | 0.267 | 1.797 |

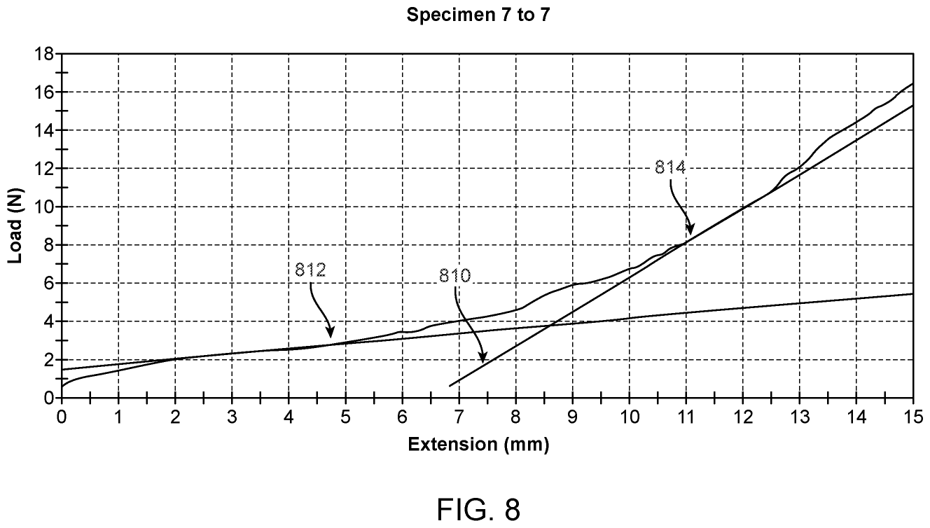

FIG. 8 shows force and displacement data 810 for subject 7. The data is shown plotted for the Load (N) versus the Extension (mm). As can be seen from the graph, force data (N) is acquired for several tissue displacement locations (mm). A first portion 812 of the data can be fit with a linear regression line to determine the slope for first amounts of loading, and a second portion of the data 814 can be fit with a second linear regression line to determine the slope for second amounts of loading, for example.

The Magnap device is currently undergoing Food and Drug Administration (FDA) approved clinical trials. Force displacement data as described herein can be obtained from subjects treated with magnetic devices such as the Magnap device and compared to efficacy data to establish force displacement data and the tissue model that corresponds to efficacy with the Magnap device. The force displacement data and tissue model as describe herein can be used to evaluate the suitability of subjects prior to treatment.

Although reference is made to using force displacement data to evaluate the suitability of subjects for treatment with a magnetic device, the presently disclose systems and methods can be used to evaluate the suitability of subjects for other therapies, such as a mandibular advancement device (MAD) for the treatment of sleep apnea.

As described herein, the computing devices and systems described and/or illustrated herein broadly represent any type or form of computing device or system capable of executing computer-readable instructions, such as those contained within the modules described herein. In their most basic configuration, these computing device(s) may each comprise at least one memory device and at least one physical processor.

The term “memory” or “memory device,” as used herein, generally represents any type or form of volatile or non-volatile storage device or medium capable of storing data and/or computer-readable instructions. In one example, a memory device may store, load, and/or maintain one or more of the modules described herein. Examples of memory devices comprise, without limitation, Random Access Memory (RAM), Read Only Memory (ROM), flash memory, Hard Disk Drives (HDDs), Solid-State Drives (SSDs), optical disk drives, caches, variations or combinations of one or more of the same, or any other suitable storage memory.

In addition, the term “processor” or “physical processor,” as used herein, generally refers to any type or form of hardware-implemented processing unit capable of interpreting and/or executing computer-readable instructions. In one example, a physical processor may access and/or modify one or more modules stored in the above-described memory device. Examples of physical processors comprise, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor. The processor may comprise a distributed processor system, e.g. running parallel processors, or a remote processor such as a server, and combinations thereof.

Although illustrated as separate elements, the method steps described and/or illustrated herein may represent portions of a single application. In addition, in some embodiments one or more of these steps may represent or correspond to one or more software applications or programs that, when executed by a computing device, may cause the computing device to perform one or more tasks, such as the method step.

In addition, one or more of the devices described herein may transform data, physical devices, and/or representations of physical devices from one form to another. Additionally or alternatively, one or more of the modules recited herein may transform a processor, volatile memory, non-volatile memory, and/or any other portion of a physical computing device from one form of computing device to another form of computing device by executing on the computing device, storing data on the computing device, and/or otherwise interacting with the computing device.

The term “computer-readable medium,” as used herein, generally refers to any form of device, carrier, or medium capable of storing or carrying computer-readable instructions. Examples of computer-readable media comprise, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

The processor as described herein can be configured to perform one or more steps of any method disclosed herein. Alternatively or in combination, the processor can be configured to combine one or more steps of one or more methods as disclosed herein.

Unless otherwise noted, the terms “connected to” and “coupled to” (and their derivatives), as used in the specification and claims, are to be construed as permitting both direct and indirect (i.e., via other elements or components) connection. In addition, the terms “a” or “an,” as used in the specification and claims, are to be construed as meaning “at least one of” Finally, for ease of use, the terms “including” and “having” (and their derivatives), as used in the specification and claims, are interchangeable with and shall have the same meaning as the word “comprising.

The processor as disclosed herein can be configured with instructions to perform any one or more steps of any method as disclosed herein.

It will be understood that although the terms “first,” “second,” “third”, etc. may be used herein to describe various layers, elements, components, regions or sections without referring to any particular order or sequence of events. These terms are merely used to distinguish one layer, element, component, region or section from another layer, element, component, region or section. A first layer, element, component, region or section as described herein could be referred to as a second layer, element, component, region or section without departing from the teachings of the present disclosure.

As used herein, the term “or” is used inclusively to refer items in the alternative and in combination.

As used herein, characters such as numerals refer to like elements.

The present disclosure includes the following numbered clauses.

Clause 1. A method of evaluating a suitability of a subject for an apnea therapy, the method comprising: placing an engagement structure on a tissue of the subject to engage the tissue of the subject; moving the engagement structure toward the tissue to a location to move the tissue; measuring one or more of the location or a force to the engagement structure at the location; and determining the suitability of the of the subject for the apnea therapy in response to the force at the location.

Clause 2. The method of the preceding clause, wherein the force comprises a plurality of forces and the location comprises a plurality of locations and wherein the suitability is determined in response to the plurality of forces at the plurality of locations.

Clause 3. The method of any one of the preceding clauses, wherein a force is measured for each of the plurality of locations.

Clause 4. The method of any one of the preceding clauses, wherein the suitability of the subject is related to a first amount of displacement at a first force and a second amount of displacement at a second force.

Clause 5. The method of any one of the preceding clauses, wherein the suitability is related to a difference between the first amount of displacement at the first force and the second amount of displacement at the second force.

Clause 6. The method of any one of the preceding clauses, wherein the suitability increases as the difference increases relative to a comparison number and the suitability decreases as the difference decreases relative to the comparison value and optionally wherein the comparison value corresponds to a statistical parameter of measured force displacement data derived from a patient population, the statistical parameter comprising one or more of a median value, an average value, or a percentile of the patient population.

Clause 7. The method of any one of the preceding clauses, wherein the suitability of the subject is related to a first amount of force at a first location and a second amount of force at a second location posterior to the first location.

Clause 8. The method of any one of the preceding clauses, wherein the suitability of the subject is related to a difference between the first force and the second force.

Clause 9. The method of any one of the preceding clauses, wherein the suitability increases as the difference decreases relative to a comparison value and the suitability decreases as the difference increases relative to a comparison value and optionally wherein the comparison value corresponds to a statistical parameter of measured force displacement data derived from a patient population, the statistical parameter comprising one or more of a median value, an average value, or a percentile of the patient population.

Clause 10. The method of any one of the preceding clauses, wherein the plurality of locations comprises a plurality of displacement locations corresponding to an anterior posterior axis of the subject and the plurality of forces comprises a plurality of forces measured at the plurality of displacement locations corresponding to the anterior posterior axis of the subject.

Clause 11. The method of any one of the preceding clauses, wherein the suitability is related to slope data of the plurality of forces at the plurality of displacement locations.

Clause 12. The method of any one of the preceding clauses, wherein the suitability increases for decreased slope as compared to an increased slope.

Clause 13. The method of any one of the preceding clauses, wherein the engagement structure comprises a recess, the recessed sized and shaped to receive tissue of the neck.

Clause 14. The method of any one of the preceding clauses, wherein the recess is sized and shaped to receive at least a portion of a laryngeal prominence.

Clause 15. The method of any one of the preceding clauses, wherein a first protrusion extends on a first side of the recess and a second protrusion extends on a second side of the recess opposite the first side of the recess.

Clause 16. The method of any one of the preceding clauses, wherein the first protrusion and the second protrusion are sized and shaped to move a hyoid bone of the subject without engaging the laryngeal prominence.

Clause 17. The method of any one of the preceding clauses, wherein the first protrusion and the second protrusion are sized and shaped to move the hyoid bone without contacting skin of the subject anterior to the laryngeal prominence.

Clause 18. The method of any one of the preceding clauses, wherein the recess is sized and shaped to be advanced posteriorly along a midline of the subject and at least a portion of the recess overlaps with at least a portion of the midline of the subject.

Clause 19. A system for evaluating a suitability of a subject for an apnea therapy, the system comprising: an engagement structure sized and shaped to engage a tissue of the subject; an extension coupled to the engagement structure; a force sensor coupled to the extension and the engagement structure to measure a force to the tissue of the subject; a movement sensor coupled to the extension and the engagement structure to measure an amount of displacement of the engagement structure; and a processor coupled to the force sensor and the movement sensor to measure the force to the tissue and an amount of displacement.

Clause 20. The system of any one of the preceding clauses, further comprising a linkage coupled to the extension to advance and retract the engagement structure.

Clause 21. The system of any one of the preceding clauses, further comprising a linkage coupled to the extension to advance and retract the engagement structure.

Clause 22. The system of any one of the preceding clauses, wherein the linkage comprises a fixed reference component coupled to a support and a movable component coupled to the extension and the movement sensor to measure displacement of the extension.

Clause 23. The system of any one of the preceding clauses, further comprises a neck support to support the neck of the subject while the engagement structure engages the tissue of the neck.

Clause 24. The system of any one of the preceding clauses, wherein the neck support is coupled to a fixed reference component of a linkage.

Clause 25. The system of any one of the preceding clauses, wherein the engagement structure comprises a recess to receive at least a portion of a laryngeal prominence and a first protrusion to engage tissue on a first side lateral to the laryngeal prominence and a second protrusion to engage tissue on a second side lateral to the laryngeal prominence, the first side opposite the second side.

Clause 26. The system of any one of the preceding clauses, wherein the first protrusion comprises a first tissue engagement surface and the second protrusion comprises a second tissue engagement surface, the first tissue engagement surface inclined at a first angle to engage tissue on the first side, the second tissue engagement surface inclined at a second angle to engage tissue on the second side.

Clause 27. The system of any one of the preceding clauses, wherein the first angle is oblique to an axis of displacement of the extension and the second angle is oblique to the axis of displacement of the extension.

Clause 28. The system of any one of the preceding clauses, wherein the first surface is inclined at the angle to correspond to a slope of a skin of the subject on the first side of the laryngeal prominence and to correspond to a second angle of a skin of the subject on the second side of the laryngeal prominence.

Clause 29. The system of any one of the preceding clauses, wherein the extension comprises an elongate axis and a linkage is configured to advance and retract the extension along the elongate axis, the elongate axis aligned with the tissue engagement structure to align the neck of the subject with the engagement structure and the axis of the extension.

Clause 30. A method of evaluating suitability of a subject for an apnea therapy, the method comprising: receiving force displacement data from tissue of the subject; and determining the suitability of the subject in response to the force displacement data.

Clause 31. The method of any one of the preceding clauses, wherein the force displacement data has been acquired with the system or method of any one of the preceding clauses.

Clause 32. A processor comprising a tangible medium configured with instructions to perform the method of any one of the preceding clauses.

Clause 33. A system for evaluating a suitability of a subject for an apnea therapy, the system comprising: an engagement structure sized and shaped to engage a tissue of the subject; an extension coupled to the engagement structure, the extension configured to couple to a removable weight; a movement sensor coupled to the extension and the engagement structure to measure an amount of displacement of the engagement structure; and a processor coupled to the movement sensor to measure the amount of displacement in response to the removable weight and record weight data associated with the removable weight.

Clause 34. The method or system of any one of the preceding clauses, wherein the engagement structure is configured to engage a skin of a neck.

Clause 35. The method or system of any one of the preceding clauses, wherein the displacement of the engagement structure is within a range from 1 millimeters (mm) to 15 mm and the force to the neck is within a range from 1 Newton (N) to 10 N.

Clause 36. The method or system of any one of the preceding clauses, wherein the tissue comprises skin and tissue beneath the skin is displaced.

Clause 37. The system or method of any one of the preceding clauses, wherein the suitability comprises a suitability parameter.

Clause 38. The system or method of any one of the preceding clauses, wherein the suitability parameter comprises one or more of scale parameter, a percentile ranking parameter, a percentile ranking parameter based on a comparison of values of a patient population, a probability of success parameter, or a pass fail parameter.

Embodiments of the present disclosure have been shown and described as set forth herein and are provided by way of example only. One of ordinary skill in the art will recognize numerous adaptations, changes, variations and substitutions without departing from the scope of the present disclosure. Several alternatives and combinations of the embodiments disclosed herein may be utilized without departing from the scope of the present disclosure and the inventions disclosed herein. Therefore, the scope of the presently disclosed inventions shall be defined solely by the scope of the appended claims and the equivalents thereof.

What is claimed is:

1. A method of evaluating a suitability of a subject for an apnea therapy, the method comprising:
placing an engagement structure on a tissue of the subject to engage the tissue of the subject;
moving the engagement structure toward the tissue to a location to move the tissue;
measuring one or more of the location or a force to the engagement structure at the location; and
determining the suitability of the of the subject for the apnea therapy in response to the force at the location.

2. The method of claim 1, wherein the force comprises a plurality of forces and the location comprises a plurality of locations and wherein the suitability is determined in response to the plurality of forces at the plurality of locations.

3. The method of claim 2, wherein a force is measured for each of the plurality of locations.

4. The method of claim 2, wherein the suitability of the subject is related to a first amount of displacement at a first force and a second amount of displacement at a second force.

5. The method of claim 4, wherein the suitability is related to a difference between the first amount of displacement at the first force and the second amount of displacement at the second force.

6. The method of claim 5, wherein the suitability increases as the difference increases relative to a comparison number and the suitability decreases as the difference decreases relative to the comparison value and optionally wherein the comparison value corresponds to a statistical parameter of measured force displacement data derived from a patient population, the statistical parameter comprising one or more of a median value, an average value, or a percentile of the patient population.

7. The method of claim 2, wherein the suitability of the subject is related to a first amount of force at a first location and a second amount of force at a second location posterior to the first location.

8. The method of claim 7, wherein the suitability of the subject is related to a difference between the first force and the second force.

9. The method of claim 8, wherein the suitability increases as the difference decreases relative to a comparison value and the suitability decreases as the difference increases relative to a comparison value and optionally wherein the comparison value corresponds to a statistical parameter of measured force displacement data derived from a patient population, the statistical parameter comprising one or more of a median value, an average value, or a percentile of the patient population.

10. The method of claim 2, wherein the plurality of locations comprises a plurality of displacement locations corresponding to an anterior posterior axis of the subject and the plurality of forces comprises a plurality of forces measured at the plurality of displacement locations corresponding to the anterior posterior axis of the subject.

11. The method of claim 10, wherein the suitability is related to slope data of the plurality of forces at the plurality of displacement locations.

12. The method of claim 11, wherein the suitability increases for decreased slope as compared to an increased slope.

13. The method of claim 1, wherein the engagement structure comprises a recess, the recessed sized and shaped to receive tissue of the neck.

14. The method of claim 13, wherein the recess is sized and shaped to receive at least a portion of a laryngeal prominence.

15. The method of claim 13, wherein a first protrusion extends on a first side of the recess and a second protrusion extends on a second side of the recess opposite the first side of the recess.

16. The method of claim 15, wherein the first protrusion and the second protrusion are sized and shaped to move a hyoid bone of the subject without engaging the laryngeal prominence.

17. The method of claim 16, wherein the first protrusion and the second protrusion are sized and shaped to move the hyoid bone without contacting skin of the subject anterior to the laryngeal prominence.

18. The method of claim 1, wherein the recess is sized and shaped to be advanced posteriorly along a midline of the subject and at least a portion of the recess overlaps with at least a portion of the midline of the subject.

19. A system for evaluating a suitability of a subject for an apnea therapy, the system comprising:
an engagement structure sized and shaped to engage a tissue of the subject;
an extension coupled to the engagement structure;
a force sensor coupled to the extension and the engagement structure to measure a force to the tissue of the subject;
a movement sensor coupled to the extension and the engagement structure to measure an amount of displacement of the engagement structure; and
a processor coupled to the force sensor and the movement sensor to measure the force to the tissue and an amount of displacement.

20. The system of claim 19, further comprising a linkage coupled to the extension to advance and retract the engagement structure.
